(19) United States
(12) Patent Application Publication
(10) Pub. No.: US 2025/0213233 A1
(43) Pub. Date: Jul. 3, 2025

(54) ONE STEP SAMPLE PROCESSING INTEGRATE-ABLE DEVICE

(71) Applicant: TECHNOCULTURE RESEARCH PRIVATE LIMITED, Patna (IN)

(72) Inventors: Sarthak TIWARY, Patna (IN); Satyam -, Patna (IN); Pritam DUTTA, North 24 Parganas (IN)

(73) Assignee: TECHNOCULTURE RESEARCH PRIVATE LIMITED, Patna (IN)

(21) Appl. No.: 18/852,679

(22) PCT Filed: Dec. 9, 2022
(86) PCT No.: PCT/IB2022/062001

§ 371 Date: Sep. 30, 2024

(30) Foreign Application Priority Data
Mar. 30, 2022 (IN) 202231019061

(51) Int. Cl. A61B 10/00; A61B 10/02; B01L 3/00

(52) U.S. Cl. CPC A61B 10/0096; B01L 3/5029; A61B 2010/0216; B01L 2200/16; B01L 2300/047

(57) ABSTRACT

The present invention discloses sample collection and processing integrate-able device comprising a sample collecting swab [1] comprising a swab tip [5], a swab handle, a swab locking slots [8] and a push-base [7]; a sample collection vial [2] comprising liquid for collection of sample [4], a linear guide for swab [9] at the base of the said vial and an unilateral lid [3] that is user friendly and enables collection and processing of a sample in one step. The device overcomes the problem of higher chances of contamination of the samples associated with use of the sample collection devices of the prior art.

### BACKGROUND

Biological samples are abundantly used for various personal and medical applications. Samples, such as blood drops, buccal, nasopharyngeal, etc. can be collected with an absorbent material such as a swab. The use of such swabs allows for non-invasive sample collection method. Swabs typically made of cotton in the form of buds are used to absorb the sample collected from the body of the object. However, most of the samples collected with the cotton buds may degenerate with time concerning the transportation time, and moreover, the multiple steps involved in handling these samples, performed in laboratories increase the chance for contamination and other possible human errors.

The conventional state of art requires a separate collection and preservation tube and further needs to be sent to a specific laboratory for further analysis. Wherein the current state of art tries to eliminate the complications involved in the whole process by reducing the steps and providing a one-step mechanism along with a pre-assembled device that does the job from sample collection to processing to detection.

The steps involved in the conventional state of art as well the complications related to the handling of the device results in the following challenges, upon which we are able to provide improvements through present invention. Mentioned below are the problems faced by the conventional state of art:

In the conventional state of art, having the swab and a sample container as separate components requires the use of carefully inserting the swab into the sample container. The process further requires the handling to be done without allowing the swab to touch any surface including the surface of the container itself.

The conventional state of art also restricts the swab from being kept on any surface including that of a tabletop in between the processes of sample collection. In order to avoid any contamination, the user needs to be extra careful which also sometimes add to the complication of the process.

The most significant problem concerning the end-user is that the users are required to follow multiple steps precisely as instructed in order to collect the sample and to collect the sample in the use and throw cartridge where the sample is further processed and analysed.

Such conditions present certain handling challenges for users with poor vision, impulsive nature, or the inability to read and understand and follow instructions relating to this process. Accordingly, an improved device for sample collection was a desideratum.

Swabs and the biological samples collected through with the help of swabs, in the conventional state of art require a separate extraction and preservation technique in the form of air drying or freezing. These processes possibly increases the chance for contamination or degeneration of the collected sample. There is a fair chance of human error since the handling requires the user to strictly follow the instructions and avoid touching the swab portion that has the sample on it. Also, the swab must not be allowed to come in contact with any surface of the device or the sample container. These complicated steps altogether makes the conventional methods for collecting the biological samples an inconvenient user product, thus declining its use in a widespread scenario. The current state of art therefore, tries to eliminate the problems faced in the conventional state of art and allows the user to comfortably handle the overall operation on its own.

### OBJECT OF THE INVENTION

The primary objective of the present invention is to reduce the required procedure steps of collecting a biological sample with the help of cotton swab into a sterile container, into a single step.

Another objective of the present invention is to constitute a single module that merges all 3 modules; a swab, a sample container, and a use-and-throw cartridge into a single assembly.

Another objective of the current state of art is to eliminate all the limitations of the conventional prior art and provide a single unit, convenient and user friendly device for use in various personal and diagnostic applications.

Yet another objective of the current invention is to provide a separate liquid chamber within a single device.

Another objective of the present state of art is to make a cost-efficient device through the minimalistic design of a one-step biological sample collection, extraction and processing device.

Yet another objective of the present device is to achieve the geometry optimization concerning the reduction of total reaction reagents as well the sample volume and ease of use for user.

Another objective of the current invention is to achieve a convenient handling of the device through the geometry of the sample container concerning the swab in such a way that placing it on flat surfaces such as a table top does not cause the swab to come in contact with any surface. This furthermore decreases the chances of contamination due to poor or relaxed handling by the end-user.

Yet another objective of the present invention is to reduce the time required for the overall process.

Another objective of the current state of art is to provide an ease of use for the users. Yet another objective of the current state of art is to provide a simple linear retraction mechanism that allows to operate the device with ease and comfort. The present invention proposes a mechanism that reduces the chance of mishandling or any confusion that could possibly be created during the operation.

Another objective of the present invention is to achieve the process of amplification and detection through the micro fluidic chip that is put together along with the detection unit.

Yet another objective of the current state of art is to provide an environment friendly option with the use of bio-degradable and non-toxic materials.

### SUMMARY OF THE INVENTION

The current state of art provides methods, systems and a single unit device for the collection, extraction of the biological sample in the form of buccal, nasal or blood DNA cells and also for further processing and detection of the collected biological samples. An embodiment of the current invention is a one-step mechanism device which allows the user to perform the sample collection and further processing in nonclinical scenario, such as the user's home. The collected biological samples can be used for various personal genomics as well as diagnostic applications.

The invention comprises a swab, a sample collection container, and a use-and-throw cartridge combined into a single component through the means of mechanisms that allow them to behave in a principled manner in relation to one another.

In an embodiment, the current state of art is a device including; a swab with a swab tip containing the absorbent material for sample collection, an extraction and processing chamber where the sample is extracted to come in contact of the liquid, which is a reagent that further helps in detection, and a reader that displays the result after the completion of the overall process.

Ease of use for the user is achieved through the assembly of the biologic sample collector made-up of a non-porous metallic or non-metallic container such as plastic or silicon. The sample collection or the liquid container keeps the chamber sterile and DNA-free prior to use. In one embodiment of the current invention, the biologic sample collection device is housed within a non-porous metallicor non-metallic container and is further connected to a lid that is used to seal the biologic sample collection and processing chamber within the present device.

Since the goal of the current state of art in some of the embodiments is to be able to achieve a passable genetic profile of the user from whom the sample is collected, the quality of the extracted DNA and its adequacy for further processing is important. The biologic sample collection device described in the current state of art enables collection of a sufficient amount of sample and also provide sufficient DNA to support further processes.

In yet another embodiment, the present invention is a method, including: the collection of the biological sample of a subject with the help of an absorptive component attached at the swab tip. Herein the method of sample collection includes contacting the tip of the swab against a specific body portion of the subject, and further transferring the sample from the swab tip to a solution confined within the device. This whole mechanism is designed so that the swab reaches the liquid chamber without touching any other surface of the device so as to eliminate the chance of any possible contamination.

Another aspect of the embodiment discussed above is aimed towards the mechanism behind the sample collection method where the sample collection container is designed to be immaculately sealed despite the incorporation of various mechanisms for opening and closing of the lid.

In a specific embodiment, as described in more detail below, the sample-collecting swab of the current invention can be pulled downwards in a linear motion. The mechanism is such that it does not allow the swab to move in the opposite direction or fallout from the cartridge unit where the collected sample is further processed and analysed. Herein, the swab and the sample container are connected through a mechanism that is able to automatically open and close the container lid such that no possible contact between the swab and the sample container is possible.

In accordance with the feature discussed above, the swab will be integrated within the cartridge wherein, the liquid chamber is placed. Once the user finishes rubbing the swab against the desired body part in order to collect the sample, a single pull mechanism will take care of the swab to get suspended right on place without coming in contact with any surface of the device. The mechanism applied herein eliminates the chance of any human error and equally reduces the time taking steps involved in the conventional state of art. Subsequently, the swab along with the front swab holder and the swab tip is locked inside the liquid chamber, allowing the back swab holder to break apart and come out of the device.

Preferably in that arrangement, to provide a simple yet well-built mechanism, the sample container chip is designed to have appropriate flat surface and have high optical clarity in order to make various optical measurements. A significant feature of the chip assembled within the current state of art is the use of a cost efficient plastic tape, which also contributes to the overall cost cutting of the current state of art, thus making it a great choice for use within a use-and-throw cartridge.

In a further development of the current state of art which is advantageous since it can be made available in significantly low cost, the device of the present invention is aimed for the well-being of the end user. The reduction of steps and the elimination of the laboratory based mechanism, makes the current state of art even better and more user friendly. The current state of art unlike the conventional methods does not include multiple handling instructions, large volumes of reagents or a need for the professional assistance which allows the user to perform the whole process on its own comfortably at home.

The sample collection device of the present invention under the present disclosure holds several advantages over currently available sample collection devices. With the aim of providing a better insight into the mechanism and advantages of the invention, few examples of practical representations thereof are described further on this document, with reference to the accompanying drawings.

### BRIEF DESCRIPTION OF FIGURES

The above mentioned objectives and descriptions, features, aspects, and advantages of the present invention will become better understood when the following detailed description is read with reference to the accompanying figures in which like characters represent like parts throughout the figures, wherein:

FIG. 1 depicts the conventional methods and the handling process as per the current invention;

FIG. 2 depicts a flowchart comparing the usability of the conventional sample collection device and the device of the present invention;

FIG. 3 depicts steps involved in collection of the sample as per the current invention which provides an insight into the opening, closing as well as the breaking mechanisms of the excess swab handle and also the mechanisms involved in maintaining a one-way linear retraction of the swab;

FIG. 4 depicts the general idea of the one way linear retraction of the swab;

FIG. 5 depicts the opening mechanism of the sealing during the process wherein the swab is pulled down after the sample is collected;

FIG. 6 depicts 6(1) shows the Swab tip sub assembly, 6(2) shows the swab handle sub assembly [6(2)], 6(3) shows the assembled view of the swab where the swab tip sub assembly and the swab handle sub assembly connect together with the help of the both swab breaking mechanism [6(1)][6(2)];

FIG. 7 depicts the total force optimization of the present invention retaining all constrains like sealing, one way linear retraction of the swab, closing of the sample container vial lid, and the breaking of the swab remain unchanged during application of single pull of the swab;

FIG. 8 depicts an embodiment of the present invention where the number of parts and assembles are minimized;

FIG. 9 depicts in an embodiment of the present invention a significant reduction of steps of human interaction compared with conventional method and existing prior art;

FIG. 10 depicts in one embodiment of the present invention a micro fluidic chip placed in the deice for sample processing;

FIG. 11 depicts the external signal and power supply module present in the device communicating with another User interface;

FIG. 12 depicts single-mold sample collection device in one embodiment of the present invention;

FIG. 13 depicts process of sample collection by a user using the single mole sample collection device;

FIG. 14 depicts single-mold sample collection device with geometry-based closing lid locking and sealing mechanism in one embodiment of the present invention; and

FIG. 15 depicts sample collection device in one embodiment of the present invention.

Further, skilled artisans will appreciate that elements in the figures are illustrated for simplicity and may not have been necessarily drawn to scale. For example, the flow charts illustrate the method in terms of the most prominent steps involved to help to improve understanding of the aspects of the present invention. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having benefit of the description herein.

### DETAILED DESCRIPTION

The current state of art relates to the design of a sample collection swab intended for the collection of biological samples or diagnostic specimens such as buccal cellular DNA, blood cells, oral fluids, genomic DNA or the like. The current methods for obtaining a sample are typically composed of at least 3 distinct and separate components; a sample collection container and a use and throw cartridge for insertion of a sample into the device where the analysis of the biological sample is to take place. The single assembly of the present invention may comprise multiple sub-assemblies to a single part. Herein, the device of the present invention allows sample collection, extraction as well the processing of the extracted sample within a single unit and that too in a one-step mechanism.

A major challenge in the field includes the identification of appropriate source material for home-based sample collection that is appropriate for medical as well as personal applications. The problems faced come both from the prior art as well as from the challenge of producing a sample collection device that is more user friendly than existing products in order to aid in the mass adoption of personalized genomics amongst people with limited to absolutely no knowledge of sensitivities inherent in clinical sample collection. This often can lead to degradation of the biological sample and thus in turn can lead to inaccurate results upon further analysis.

The sole purpose of the present invention is to resolve one or more of the problems encountered in the prior art. As a solution to these problems, and also to achieve certain advantages which will be evident from further description, the present invention proposes a unique device for collecting as well as processing biological samples. The conventional state of art require multiple steps for performing sample collection and extraction, which makes the whole process very complicated. Also, the user have difficult time understanding and analysing and then finally performing the required steps in order to achieve the desired results. Further the conventional state of art, the whole process typically require multi-step actions since the collection of the biological samples until its analysis at a laboratory. Thus the conventional state of art lacks the scope for fully contamination free operation of the collected biological samples since it requires multiple steps that has to be conducted in the laboratory. The involved lab steps in the conventional state of art increases the chance of human error.

In the conventional state of art there is huge requirement of multiple steps, high volume and human controlled handling during the overall mechanism. The procedures of sample process and analysis require well equipped labs as well as significant time to perform steps like-drying, cutting and effective dispensing of the remaining absorbent materials. In the conventional state of art, the involved lab procedures are human controlled which increase the chance of contamination.

The conventional state of art is usually limited to a sample collection device or a sample collection and extraction device only. Within the conventional state of art, a single unit device that performs all the required steps in an easy one step method can't be achieved. Also the conventional state of art may include a micro fluidic chip that is high in cost since it is made from costly materials. Another problem with the conventional state of art is that the required volume for the biological sample and the reagents required for the reaction process in order to operate the mechanism is usually higher and even costly. The conventional state of art includes multiple steps and instructions that sometimes troubled the user and also results in incorrect handling of the device. Further in the conventional state of art usually, the collected sample is not analysed immediately upon collection and therefore increases chances of contamination or sample degradation.

The device of the current invention constitutes a swab, a sample collection container containing fluid medium or solvent, a use & throw cartridge and a sample processing device combined into a single component. The current state of art is a robust device involving one-step sample collection, extraction and processing of the biological samples through a single pull that can be further used for various personal, clinical as well as diagnostic testing. Also, the current invention shows a limited use of the components and the reduced operational steps which altogether helps in achieving a highly affordable cost of the present one step sample collecting, extracting and processing device for collection of biological samples. The current invention collect the sample with the swab and further extract the sample during which there is no chance of contact with any other surface thus, eliminating the chance of any human error. The reduced no. of steps, a quick and convenient handling of the device allows the user to perform the whole operation without facing any challenges. An easy one-step mechanism reduces the scope for the lack of understanding from the user end on how to operate the device.

The device of the current invention allows the user to accomplish the task in fewer but not more than 3 steps, which evidently provides the current state of art an advantage over the conventional sample collection methods.

In addition, in the current invention the micro fluidic chips are made with the help of cost efficient plastic tapes to bring down the cost by using a cheaper alternative technically sound alternative. Since the micro fluidic chip is a use-and-throw component, the achieved price point in the current invention is convenient to be used as a use-and-throw device. In the conventional state of art, the more no. of steps as well the separate units for collection, extraction and processing of the biological samples make it a long, time-taking process.

In the current state of art the total volume of the reagents and sample is kept very low which ultimately reduces the size of the device. Also, the significant reduction in the steps involved during the operation of the current device is achieved through the one-step mechanism of the current state of art. The current state of art successfully overcomes the problem of using multiple steps, thus making it a faster and convenient mechanism. The reduction in the volume as well the steps involved ultimately results in the reduction of cost of the invention. The current invention therefore, has a great advantage over the conventional ones. In the current state of art, the reduction of the total steps to be performed during the overall procedure allows to achieve an easy and uncomplicated mechanism.

The present invention utilizes a special combined mechanism which maneuvers between the responsibilities traditionally allotted to various different components specifically the opening mechanism, the closing mechanism and a method for sealing of the container vial and the mechanisms involved in maintaining a one-way linear retraction. The achieved design is such that it enables only one way movement of the swab which also eliminates the fear of falling or reverse pulling or pushing during the preferred mechanism. The device has one way linear retraction swab allowed to move in one axis through a liquid container containing sample with the help of guiding rails, snap fits and holes. Further the current state of art enables the user to get the results right at their home, without involving any other person or device. This makes the current state of art a unique, inventive, and truly a point of care device. The sample container of the device includes a micro fluidic chip to process biological sample and to conduct bio sensing on collected sample.

In an embodiment the current invention refers to a connection system for a use-and-throw cartridge wherein the collected sample is further analysed for the detection. The cartridge is further to a cartridge reader that displays the final obtained result. The cartridge unit also has a locking mechanism wherein the swab detaches and the extra swab holder breaks automatically (with a manual pull) at the bottom of the sample collection container while the remaining swab with collected sample is settled inside for further process.

The in-built cartridge has a liquid chamber which also is a collection chamber that allows the insertion of the sample collection swab into the cartridge. This liquid chamber is located at a fixed location within the device, wherein the swab or the absorbing pad undergoes a chemical reaction, thereby transferring a predetermined volume of the collected sample in the collection chamber.

In an embodiment, the current state of art is a device, comprising: a swab including an absorbent material attached to the swab head, an extraction chamber for receiving the swab and extracting the sample collected on the swab. The collection of the desired sample is done through the swab with the use of just one hand and without any chance of contamination. Inside the collection chamber is a pre-defined volume of reagent that further allows the extraction of the collected biological sample to help in detection to finally result in displaying a positive or negative result after all the procedures have been carried out successfully. Further, the current state of art enables the user to get the results right at their home, without involving any other person or device. This makes the current state of art a unique, inventive, and truly a point of care device. The sample container of the device includes a micro fluidic chip to process biological sample and to conduct bio sensing on collected sample.

Unlike the conventional state of art, the current invention does not require large volumes or raw materials due to the single unit device and a one-step mechanism.

State of the art in sample container vials depend on some form of an opening mechanism involving a human hand coming in direct contact with the opening section of the container vial.

These opening mechanisms are comprised of but not limited to the use of snap-fit caps, pressure fit caps, screw-on caps, plastic seals based on adhesives, heat, ultraviolet ray-based sealing, elastomer-based sealants, among other similar sealing mechanisms.

The human hand must in the process of opening these vials, come in contact with container vial surfaces along with the need to apply forces onto the opening section.

These opening mechanisms hence produce usability challenges for users with physical disabilities such as essential tremor disorder, fidgety hands, etc.

In a conventional scenario the opening section of the container vial containing liquid, creates a possibility for contamination due to direct contact with air because of the large opening.

Furthermore, the need to come in contact with the opening section of the container vial creates a possibility for contamination of various kinds such as deposition of oily compounds, dirt, dust, cellular debris, and microorganisms from finger surfaces.

Along with the above mentioned challenges associated with the state of the art in opening mechanisms involved in sample collection, it would be preferred if the need for such a step involving an explicit opening of the container vial can be completely removed.

Furthermore, in some embodiment's needing prefilled liquids to be contained within the container vial throughout its product life cycle—the sealing must be able to withstand various environmental scenarios witnessed in the process of transportation and logistics handling without leading to leakage that could disturb the utility of the product. Removal of the need for the human to explicitly open the container vial would reduce at least one set of instructions regarding usage and precaution to be eliminated from user manuals and other user guidance communication materials.

However, automating the opening process, would in conventional scenarios require the use of additional parts, in order to facilitate the opening as a reaction to another movement or action carried out directly or indirectly by the user.

Similarly, State of the art in sample container vials depend on some form of a closing mechanism involving a human hand coming in direct contact with the opening section of the container vial.

These closing mechanisms are comprised of but not limited to the use of snap-fit caps, spring loaded, complaint mechanism based, pressure fit caps, screw-on caps, plastic seals based on adhesives, heat, ultraviolet ray-based sealing, elastomer-based sealants, among other similar sealing mechanisms.

The human hand must in the process of closing these vials, come in contact with container vial surfaces along with the need to apply forces onto the closing section.

These closing mechanisms can hence produce usability challenges for users with physical disabilities such as essential tremor disorder, fidgety hands, etc.

The human hand must in the process of closing these vials apply an adequate amount of force to close the closing section which may and may not close the vial in its most complete form—depending on the force applied—leaving room for leakages.

Furthermore, the container vial, while closing, creates a possibility for contamination of various kinds such as deposition of oily compounds, dirt, dust, cellular debris, and microorganisms from finger surfaces carried from the opening section.

The container vial, while closing, creates a possibility for leakage due to physical obstructions upon sealing mechanism.

Along with the above mentioned challenges associated with the state of the art in closing mechanisms involved after the sample collection, it would be preferred if the need for such a step involving an explicit closing of the container vial can be completely removed.

Furthermore, in some embodiment's needing prefilled liquids to be contained within the container vial throughout its product life cycle—the sealing must be able to withstand various environmental scenarios witnessed in the process of transportation and logistics handling-without leading to leakage that could disturb the utility of the product.

Removal of the need for the human hand to explicitly close the container vial would reduce at least—one set of instructions regarding usage and precaution to be eliminated from user manuals and other user guidance communication materials.

However, automating the closing process, would in conventional scenarios require the use of additional parts, in order to facilitate the closing as a reaction to another movement or action carried out directly or indirectly by the user.

State of the art in sample container vials may depend on some form of one way linear retraction mechanism incorporated for positioning, detection and locking of swab or any moving parts incorporate within the container vial involving the use of the human hand coming in direct contact with the entire body section.

These one way linear retraction mechanism incorporated for positioning, detection and locking of the swab are composed of but not limited to the closing section, sealing section, swab locking section, opening section, swab breaking section, pressure fit caps, screw-on caps, threaded caps for opening, positioning, etc.

In the state of the art, the one way linear retraction mechanism incorporated for positioning, detection and locking of the swab requires a set of instructions in order to perform the task. However, the instructions are not specified as per requirements causing problems such as but not limited to misalignment, dislocation, or over-tightening.

The one way linear retraction mechanism incorporated for positioning, detection and locking for swab concerning the state of the art, therefore, present certain usability challenges for users with physical disabilities such as essential tremor disorder, fidgety hands, etc.

The one way linear retraction mechanism incorporated for positioning, detection and locking for swab designs might have sharp corners or edges where the possibility for contamination of various kinds such as aging of oily compounds, dirt, dust, cellular debris, and microorganisms from finger surfaces takes place.

Along with the above mentioned challenges associated with the state of the art in one way linear retraction mechanism incorporated for positioning, detection and locking for swab involved in sample collection, it would be preferred if the need for such a step involving an explicit one way linear retraction mechanism incorporated for positioning, detection and locking of the swab can be completely simplified for user.

One way linear retraction mechanism incorporated for positioning, detection and locking for the swab needs number of assembly or sub-assembly. Miniaturized the mechanism and incorporate within the device is challenging.

However, automating the one way linear retraction mechanism incorporated for positioning, detection and locking of the swab process, would in conventional scenarios require the use of additional parts, in order to facilitate the positioning, detection and locking as a reaction to another movement or action carried out directly or indirectly by the user.

State of the art in sample container vials depend on some form of sealing mechanism for providing sealing for the sample container vials that have the liquid inside of it. The state of the art for the sealing of sample container vials containing liquid using the conventional sealing mechanism is not limited to threaded sealing cap, threaded cap, snap fit caps which may not cause adequate sealing because of air voids, micro particles, etc. present on the mating slot.

The liquid pre-filled inside the sample container is not stable, therefore, while opening the container for inserting the sample inside the liquid chamber, there is a possibility that the liquid may fall out of the container. Such a scenario appears due to certain vibrations, unstable handling or mishandling of the de-vice during opening of the sealing lid, or during the wrong orientation while inserting the sample collection swab. The presence of air voids within the sealing section, may cause the problem of leakage during the opening of the sealing lid. Due to this leakage and the possibility of the liquid coming in contact with the other parts of the device, outside of the sample container vials, the chance for cross contamination is increased.

In the state of the art, the liquid inside the sample container vials may get stuck between the sealing section and the vial body such as on the sealing cap where threaded sealing has been done.

In the conventional devices, a poor sealing may cause the liquid present inside the sample container vials to evaporate.

In the state of the art, however the plastics do not have porous properties but, the liquid filled inside the vials may react with the plastic that has been used for the manufacturing of the vials. While in contact with air due to porous sealing mechanisms not limited to threaded sealing cap, threaded cap, snap fit caps of the vial.

In the state of the art, the sealing of the sample container vial is usually made from but not limited to silicon which may cause stiff friction at the sealing mechanism. From the user point of view such an experience might not be pleasant.

The conventional sample container vials do not have any other assembly including the sample collecting swab, sample collector tip, and the sample collector attached in a single assembly. Since multiple assemblies require multiple sets of instructions for conducting the procedure, from the user point of view such an experience might not be pleasant and would be rather confusing. Such assemblies might also require several guidelines in order to avoid contamination as well as to avoid wastage of the liquid present in the container vial, significantly in scenarios where the container vial does not have an adequate sealing mechanism to avoid leakages.

Stability to the liquid inside the container vials can be provided by adequate sealing to the opening lid. If the container vial acts as a single assembly where the sample collecting device and the sample container vial contain liquid are sealed together with the help of any sealing mechanism. This kind of assembly can reduce the no. of steps as compared to prior art.

The sealing mechanism on the combined assembly may be required to apply a friction force while contact with the moving parts. Sometimes the contact of that sealing acts like a stiff body causing friction. Such friction force generated during the mechanism may provide a bad experience to the user.

This present invention solve problems with respect to conventional as well as existing prior art issues. Some secondary problems may arrive while designing and solving the primary problems. Possible solution of each problem discussed in detailed with diagram below.

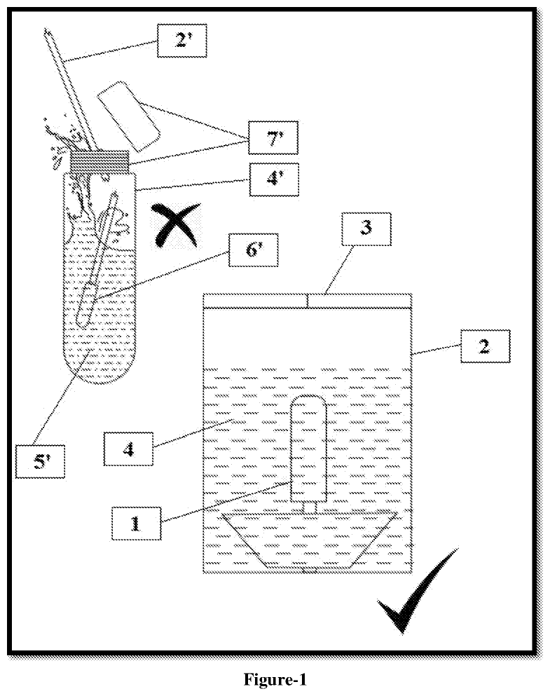

FIG. 1 shows the conventional methods and the handling process as per the current invention. The state of the art lacks the assurance for a contamination free handling of the swab due to the long set of manual steps involved in the process. Wherein, in the current invention the in-built mechanism enables a contamination-free handling of the swab therefore, making it ideal for use by user.

In the state of the art, the conventional sample collection swab and the conventional sample collection vial comes as a separate unit which makes it difficult for handling. Moreover, the user finds it difficult to put the conventional swab inside the container without letting it touch any outer part of the container. This process requires a lot of concentration and careful handling of the conventional swab.

In FIG. 1 shows the conventional sample collection swab [2′] and the conventional sample collection vial [4′] comes into contact during the process of collecting the sample by a user. In generally the conventional swab tip [6′] comes in contact with the sample collection vial [4′] during the process of collection of sample as shown in FIG. 1.

Ideally the conventional sample collection vial [4′] has liquid [5′] contained in it and sealed with help of conventional vial sealing mechanism [7′] combining lid and neck of the bottle. The liquid contained in the conventional sample collection vial [4′] may spill out during handling and also because of the large opening mouth of the conventional vial [4′].

In the current invention shown in FIG. 1, the swab [1] and the sample collection vial [2] are assembled within a single device. This not only reduces number of steps required in the handling of the device but, also increases the ease of use for the user. The mechanism of the present invention is such that the swab [1] is pulled inside through unilateral lid [3] opening inside the sample vial only, instate of such as a threaded seal cap of the vial with liquid [4] where the sample is finally collected for further processing.

Opening mechanism, closing mechanism and a method for sealing of the container vial and the mechanisms involved in maintaining a one-way linear retraction automated in this invention explained further. The current invention therefore, provides an advantage over the state of the art, making it more user-friendly. The reduction of steps and extra components also leads to the cost-reduction of the overall device.

Moreover the sealing mechanism is such that it is able to withstand various environmental scenarios witnessed in the process of transportation and logistics handling—without leading to leakage that could disturb the utility of the invention.

One of the novel objective of the invention is to reduce the number of operating or usability steps involve in existing prior art or conventional methods. Some of the steps such a step involving an explicit opening, closing, detachment of swab of the container vial can be completely removed while not effecting any constrains involve on this.

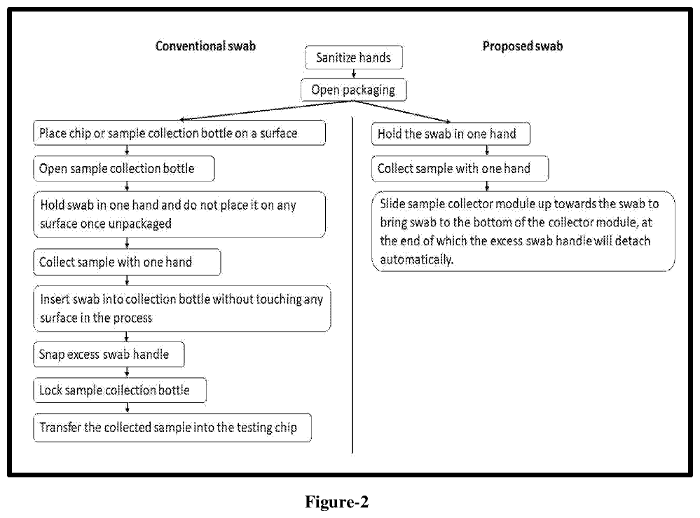

In FIG. 2 shows a flowchart comparing the usability of the conventional sample collection device and the device of the present invention. The conventional sample collection is a time taking procedure comprising around ten or more steps whereas, the current invention consists of five or fewer steps.

The handling of the swab is considered as the complicated part as it requires to be kept contamination free throughout the whole procedure from holding and collecting the swab with one hand and inserting it directly into the collection bottle without letting it come in contact with any surface.

The conventional method of the sample collection requires that the user follow several steps in order to conduct the sample collection as shown in FIG. 2. The conventional steps comprises but not limited to the steps elaborated further. Before starting the operation the user needs to sanitize his/her hands. The sample collection bottle is then opened and placed on a surface. The swab is then used for collecting the sample from the body part of the user. This sample collection process is extremely complicated as the swab strictly requires to be kept contamination free. The swab is then inserted into the sample collection bottle without allowing it to touch any surface throughout the whole process. The concluding steps require snapping of excess swab handle, locking the sample collection bottle and finally transferring the collected sample into the device.

The conditions related to the conventional methods of sample collection present certain handling challenges for users with poor vision, fidgety hands, or the inability to read and understand and follow instructions relating to this process.

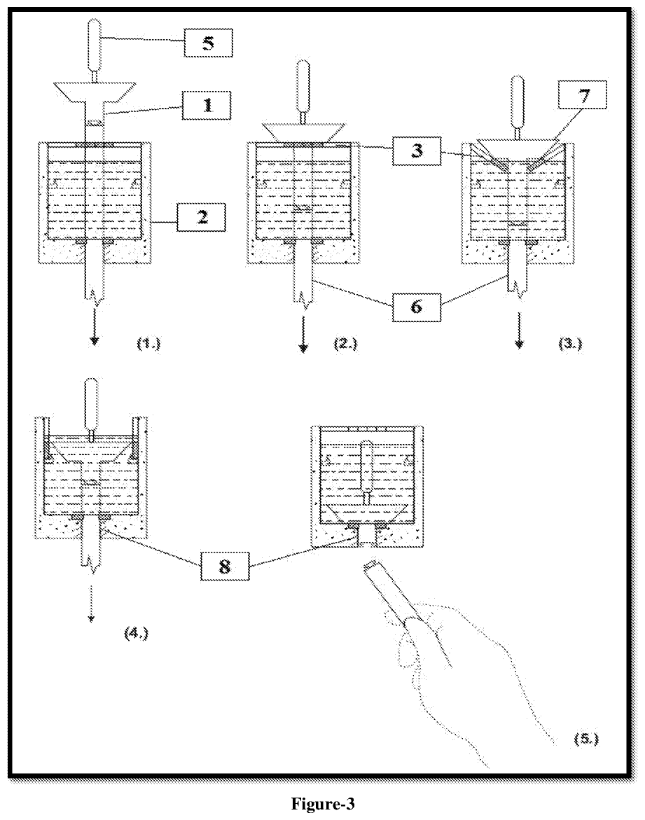

The current invention herein, reduces 10 or more steps into a procedure comprising but, not more than 5 steps. Users need to sanitize their hands before opening the package. The User then opens the lead cover and hold the swab with the help of one hand and collect sample. Once the sample is collected, the user slides the sample collector module up towards the swab in order to bring the swab to the bottom of the collector module, wherein the excess swab handle automatically detaches. The current invention therefore, aims to make the complicated sample collection devices more user friendly and innovative. FIG. 3 shows the steps involved in collection of the sample as per the current invention which provides an insight into the opening, closing as well as the breaking mechanisms of the excess swab handle and also the mechanisms involved in maintaining a one-way linear retraction of the swab. The goal of the invention is to maintain simple, for an easy user experience during using of the device as well as designing a cost effective device. The simplified steps design is explained further with keeping all constrains.

FIG. 3(1) to FIG. 3(5) is showing 5 state of a simple illustrative view of the sample collection process. The single device comprising a sample collecting swab [1], a sample collection vial with liquid [2] contained inside. The sample collecting swab [1] comprises but is not limited to swab tip [5], attached to swab handle [6] and push-base [7] attached to the swab handle that enables opening of the unilateral lid [3]. The sample collection vial [2] comprises but is not limited to a one way linear retraction mechanism [8] and container unilateral lid [3].

FIG. 3(1) shows the initial state of the device where the user can take the sample through the swab tip [5]. The next step is pulling of the swab by applying some force by the user. FIG. 3(2) to FIG. 3(5) shows the different state of the device. During the pulling, the push-base [7] hit the container unilateral lid [3] and pushes it open. The swab moves linearly downward with help of linear retraction mechanism [8]. The swab tip enters inside the container vial without touching any external or internal surface including the walls of the sample container vial [2] as well as the sample container unilateral lid [3]. The unilateral lid [3] snap close when the swab [1] reaches the position where the swab tip [5] is completely submerged within the liquid present inside of the container vial. Finally the excessive swab handle breaks automatically.

Design of such a device requires several mechanism to be performed such that all task is completed in a single force application. The mechanism should be miniaturized so that it can fit inside of that device and also the device should be cost-effective.

To avoid the contact of the push-base [7] with the sample container unilateral lid [3], a sensing mechanism is needed which can sense the position of the swab. The challenges to arrive at the inventive design with solutions are explained further in detail.

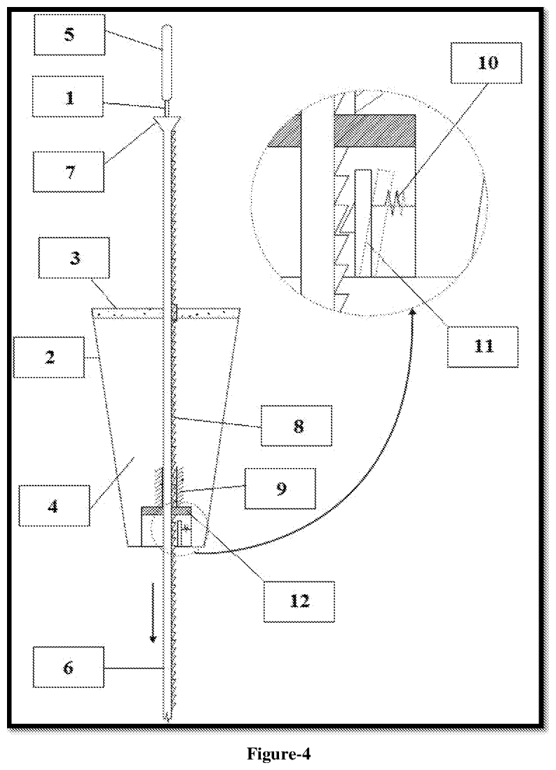

In FIG. 4 shows the general idea of the one way linear retraction of the swab. In conventional method the swab can move in multi direction which can cause failures of the process by contaminating the sample. Multi-directional movement of the swab may allow the swab to come in contact of physical surface and or any surface that may be external to or internal of the sample collecting vial.

One way linear retraction of the swab can solve the problem. FIG. 4 shows the general idea of the one way linear retraction of the swab. Sample container vial with liquid inside [4] comprise a locking and one way linear retraction mechanism [8] solves this problem. The objective is to completely submerge the swab tip [5] in the liquid present inside the sample container vial [2]. The invention show the mechanism by which the sample collecting swab [1] can pass through the sample container vial [2] with all constrains remaining same or unchanged.

The sample collecting swab [1] comprise but not limited to a push-base [7], a swab tip [5], a swab handle [6], swab locking slots [8]. The push-base [7] is used for opening the unilateral lid [3]. The linear guide for swab [9] present in sample container vial [2] does not allow to rotate the swab along its axis. This keeps the sample collecting swab to linearly retract. The wall of the linear guide for swab [9] is restricted to break the linearity constrain and sticks with the sample collecting swab [1]. The linear guide for swab therefore prevents the swab tip [5] from touching and coming in physical contact inside or on the exterior surface of the sample container vial [2]. Excess Friction force between sample collection swab [1] with linear guide of the swab [9] can cause movement restriction of the swab handle [6] To solve this. surface treatment and choice of material is important. The materials like Polytetrafluoroethylene (PTFE), Polyimide (PI), Polyether ether ketone (PEEK), Polyphenylene sulfide (PPS), Nylon, Polyoxymethylene (POM) and Polyester indicates their use for tribology (friction, lubrication and wear) because of their good sliding frictional properties.

Among all low friction plastics materials, the fluoropolymer (PTFE) have lower value of coefficient of friction and hence is a good choice for sliding applications. In case of PTFE, there is no need to add any fillers or additives to improve its frictional characteristics. In addition, PTFE show good performance, especially in the environments, which will expose to the part of chemical stress.

Pure polymers such as PTFE and linear polyethylene are the self-lubricating polymeric materials with high intrinsic lubricity. In other polymeric materials but not limited to Polyimide (PI), Polyether ether ketone (PEEK), Polyphenylene sulfide (PPS), Nylon, Polyoxymethylene (POM) and Polyester, composites are formed with the introduction of either lubricants or nano-sized additives.

Surface treatment by using different coatings like graphene-like material, molybdenum disulfide (MoS2), Poly(phenylene sulphide) and Polydopamine coatings is also possible with the thermoplastics. The coating application on polymer surface is not limited to the use of different tribological coatings such as PTFE, PEEK, polyethylene (PE), ultra-high molecular weight polyethylene (UHMWPE), Epoxy, Polyimide, Polydimethyl siloxane (PDMS), and Polymethylmethacrylate, PMMA.

Linear retraction may be carried out by implementing linear guide for swab [9]. However, backward and a forward movement of the sample collection swab [1] is still possible. The swab tip [6] may touch the container unilateral lid [3] which can lead to contamination and the primary constrains breaks.

The sample container vial with liquid inside [4] comprise but is not limited to methods of locking one way linear retraction mechanism [8]. This locking in one way linear retraction mechanism [8] comprises but not limited to a locking key bar [10] which is connected to a locking spring [11], and some sealing material [12] preventing leakages of the liquid present inside of the sample container vial [2]. The mechanism works similar to cable tie mechanism. The locking spring [11] allows the locking key bar [10] to react as flexible body. The locking key bar [10] locks the sample collection swab [1] when in contact with swab locking slots [8]. The combined design mechanism allows the sample collection swab [1] to move forward but restrict in backward movement. By the said mechanism the invention retain it's all constrains and enables less or no physical contact on swab entry by one way linear retraction.

Sharp corners and edges present in the swab locking slots [8] may cause contamination, also that's leads to cause leakages of the liquid present inside of the container vial. Since one of the objective of the invention is to prevent contamination when swab enters the liquid containing sample container vial so that no contact is made with any external or internal surface with the swab tip [6]. The container unilateral lid is the critical part of the device where the physical contact may happen.

The swab [1] cut's through the sample container vial with both end, therefore sealing of the both end is important to prevent possible contamination.

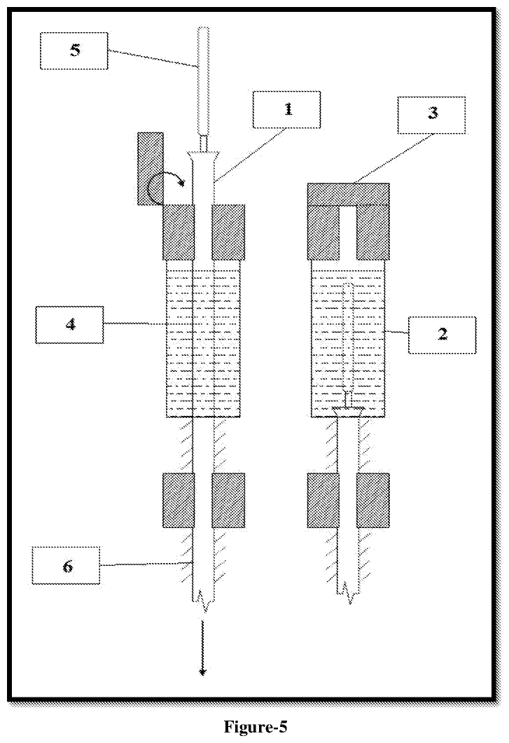

FIG. 5 shows the opening mechanism of the sealing during the process wherein the swab is pulled down after the sample is collected. Herein, the swab handle pierces the sealing and slides through it thus, opening the sealing for the entry of the swab. The pulling mechanism is controlled through a one-way linear retraction movement of the swab and the force applied by the user. The swab tip enters the liquid chamber [4] after being pulled down, by the user.

Sealing of the container unilateral lid [3] may be difficult. The sealant of the container unilateral lid [3] must seal the container containing the swab [1]. Also, after closing, the sealant must completely fill the cross-sectional area of the swab [1] when stored.

The design of the swab therefore must be optimized and particularly the swab diameter should be such that it does not touches any of the surface during the process of sample collection. For best result that swab tip must capture/absorb optimal amount of sample. Therefore, the swab tip diameter should have high surface area.

One of the objective of the invention is the cost reduction of the device. So that the design needs to be as compact as possible and should not have unnecessary volume. Therefore the volume of the liquid inside of the sample container vial should not be more than the required amount. Again the total surface of the swab tip must submerge completely into the liquid inside of the sample container vial in order to completely extract the biological sample collected in the swab.

The problem is therefore to increase the diameter and the length of the swab tip which may lead to physical contact of the swab tip with the sample container vial. Also the sealing of the entire sample container vial needs to be constrained completely.

Referring to FIG. 5, the Swab tip diameter ideally should be less than the diameter of the upper sealing opening diameter and less than the diameter of the swab handle such that the swab tip must not come in contact with any physical surface during the sample collection or during the sealing of the sample in the collection vial. This can significantly reduce contamination when as compared to the conventional method and existing prior art.

FIG. 5 shows an embodiment where the swab is being pulled down by an user after sample collection. The swab have two section, one is the swab tip [5] and another is the swab handle [6]. Diameter of the swab tip is just less than the diameter of the swab handle. The sample container vial [2] contain liquid [4] inside of it. The volume of the liquid inside of the vial is optimized to ensure complete submerging of swab tip [5] and no leakage. In this embodiment sealing is provided by the implementing sealing material.

In general the swab [1] is contained within the sample container vial initially, the swab handle [6] acts like a wall where the sealing material comes in direct contact of the surface and fills gap. The diameter of the opening of the sealing is designed such that the swab tip [5] must not touch the sealing surface when its being pulled down as shown in FIG. 5. Same sealing method is applied for the lower side of the swab container vial [2].

The unilateral lid [3] of the sample container vial [2] also have a layer of sealing material which comes in vertical contact with the sample container vial [2]. The sealing material opens when the swab tip reaches inside the sample container vial. The closing mechanism sense the swab tip [5] position and seals from top of the sample container vial. The elastic sealing material may be made of, but not limited to, silicon rubber, hard silicon, soft silicon, rubber, etc., to provide reliable sealing. The sealing material also need to be compatible with the swab handle material for maintaining adequate sealing. The material used in swab handle is disclosed further.

In still another embodiment shown in FIG. 5 the opening mechanism in conventional being completely eliminated by the design of the present invention, such that the problems associated with the opening of the sample container vial [2] will be completely eliminated. To the best of our knowledge, no such mechanism exist in prior art. The improvement can easily eliminate the problem of contamination existing in prior art.

Another objective of the present invention is to break the excess swab after the closing of sample container vial unilateral lid [3] and therefore a cutting or breaking mechanism needs to be in place. Application of the excessive force to do the needful is avoided. Also, swab must not lose the linearity as well as the sealing of the container vial.

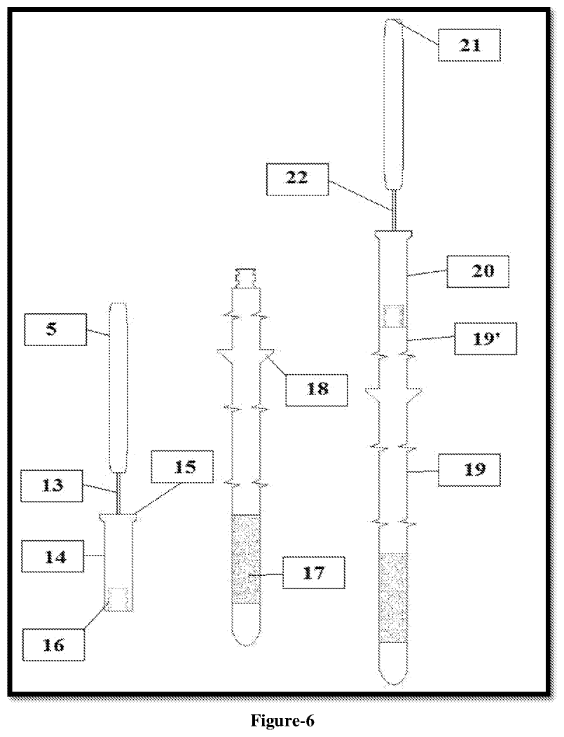

In another embodiment FIG. 6 shows swab where the swab design overcomes the problems mentioned before. According to the design consideration the modules comprises but not limited to the swab tip sub assembly and the other one is swab handle sub assembly. The two parts are connected with each other and the breaking mechanism is enabled at this joint. The breaking mechanism comprises but not limited to snap fit, cutting notch, thin joint, glued, press fit, threaded, etc. Detailed description of this embodiment is discussed further.

FIG. 6(1) shows the Swab tip sub assembly [6(1)] comprises but not limited to a swab tip, a swab collector flexible linkage [13], and a front swab handle [14]. The front swab handle [14] further comprises but not limited to front swab detecting and locking slot [15], and front swab breaking mechanism [16]. FIG. 6(2) shows the swab handle sub assembly [6(2)] comprises but not limited to back swab handle, swab gripper [17], retraction notch [18], and back swab breaking mechanism [19]. FIG. 6(3) shows the assembled view of the swab where the swab tip sub assembly and the swab handle sub assembly connect together with the help of the both swab breaking mechanism [6(1)][6(2)].

In FIG. 6 shows a swab embodiment where the diameter of front swab handle [19] and the diameter of the back swab handle [20] is equal. The diameter of the swab tip [21] is less than the both diameter of the front swab handle [20] as well as back swab handle [19] so that the swab tip does not make any physical contact while pulling the swab into the sample container vial as mentioned earlier. The swab tip [5] and the front swab handle [14] is connected to swab collector flexible linkage [13]. The diameter of the swab collector flexible linkage is smaller than the swab tip diameter [21].

The front swab section (swab-tip) can be made from different materials but not limited to nylon-flocked, cotton, rayon (viscose), small and large foam (polyurethane), knitted polyester and calcium alginate, etc. The swab handle sub assembly too can be made from different materials such as plastics (polystyrene (PS) and polypropylene (PP)), paper (more basic), wood (birch trees) and metal wire (aluminum). The sterile wood shaft are best and budget-friendly selection of choice for basic applications. In plastics, polystyrene is generally preferred, but polypropylene is also used because of its higher resistant to breaking and increased level of durability. Among metals, aluminum is used because of its malleability.

The swab tip [5] design must be optimized so that it can collect maximum possible sample from the body part of the object. The hydrophilic open-fiber morphology could be considered for swab tip design to boost sampling. Different substrates, including nylon-flocked are useful but not limited to cotton, rayon, polyester, calcium alginate and foam materials. The performance of the swab sampling is expressed in terms of its extraction and recovery efficiencies.

Swabs may have higher collection efficiencies, but show poor performance upon cell release. This performance may vary from material to material, but not limited to nylon-flocked, cotton, rayon, polyester, calcium alginate and polyurethane foams. Moreover, the absorption capacity is closely related to the swab morphology, which can be easily analyzed by Scanning Electron Microscopy (SEM). Each swab have different morphologies. This is the reason that the variation in the extraction and recovery efficiencies of swab could be found. The better performance can be obtained for tightly wound, knitted, reticulated flocked fibers.

The overall shape, length, size of the swab is optimized and designed in a way that it is functions according to the criteria and constraints of the current invention. A retraction notch [18] is placed on the swab handle sub assembly which restrict the swab to move backwards so that the swab does not make contact with surface of the sample container vial [2] or the surface container vial unilateral lid [3] if mishandled by the user as shown in FIG. 6. The problem that the swab can move linearly forward and backward starting from the retraction notch [18] is handled by another modifications.

In FIG. 6(3) shows the swab tip sub assembly [6(3)] having a swab detecting and locking slot [15] which is incorporate to restrict both way linear retraction. This swab detecting and locking slot [15] designed and optimised such a way that whenever a force is applied by the user, the swab is pushed inside the sample container vial and the swab tip gets completely submerged in the liquid, the swab detecting and locking slot [15] locks at the bottom surface of the sample container liquid storage chamber. The design of the swab detecting and locking slot [15] is optimized such that it is tightly clamped or locked at the bottom surface of the sample container liquid storage chamber. Thus, the motion gets restricted between the swab detecting and locking slot [15] and the retraction notch [18]. Further to restrict the swab movement forward and backward between the swab detecting and locking slot [15] and the retraction notch [18] is in place, the swab must break whenever the swab detecting and locking slot [15] touches the bottom surface of the sample container liquid storage chamber without breaking the constrains like sealing and the one way linear retraction.

The device design is so optimized that whenever an adequate force is applied by the user on the swab gripper [17] and pulled, it leads to breaking of the friction force between sealing component with the swab body, the friction wall responsible for linear retraction, the locking force that is required to lock the swab between swab detecting and locking slot [15] at the lower surface of the sample container liquid storage chamber, and results in detachment of the swab in two parts while maintaining the sealing.

When the swab detecting and locking slot [15] reaches the bottom surface of the sample container liquid storage chamber, the embodiment needs to be sense the position and the container vial unilateral lid should close with closing mechanism. The mechanism can be controlled by but is not limited to mechanical, electrical or electronic methods. The solution of the closing mechanism of the sample container vial lid considering all constraints is further explained.

The experience of the user needs to remain unchanged with respect to the pulling force applied, so the pulling force required to complete the total process needs to optimized. Also the objective is to retain all constrains like sealing, one way linear retraction of the swab, closing of the sample container vial lid, and the breaking of the swab, which should remain unchanged.

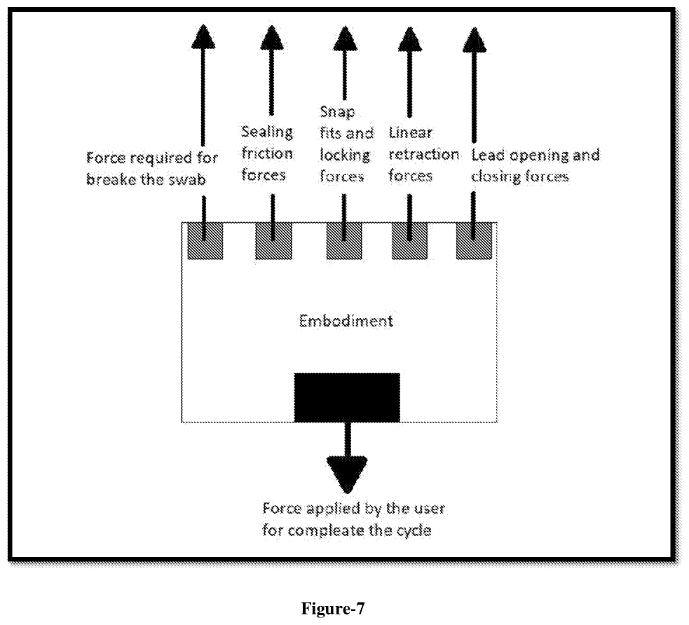

In FIG. 7 shows the total force optimization of the present invention so that the objective to retain all constrains like sealing, one way linear retraction of the swab, closing of the sample container vial lid, and the breaking of the swab remain unchanged during application of single pull of the swab.

One of the embodiment mechanism comprises but is not limited to a swab, an inner liquid containing vial module and an outer triggered housing module. Each and every module, sub-assembly and component together represent the one step sample processing integra table sample processing device.

The swab after it has been pulled down by the user by applying adequate amount of force on completion of sample collection. The force applied by the user on the swab need to be greater than but not limited to the summation of the linear retraction force which is caused by the one way linear retraction mechanism along with the wall friction forces; both top and bottom sealing material forces which is indirect contact with the swab to prevent the leakage; springs for pulling down the sample container vial, snap fits and locking forces on the sample container vial with the outer triggered housing, as well as the locking force applied on the detection and locking slot with the sample container liquid storage end; closing of the sample container lid forces as well as the opening of the lid if required; and the force required for breaking the swab as shown in FIG. 7.

Choosing the material of the swab, the sealing material, the dimension of the snap fits, stiffness of the spring, clearance between the wall of the linear retraction mechanism, overall dimension of the sample container vial as well as the outer triggered housing, design of the swab breaking mechanism, clearance between friction walls, surface treatment of the surface for reducing friction forces, etc., can be simulated by applying trial and error prototyping and optimized. So the single pulling force applied by the user on the swab for complete the cycle can be defined and optimized.

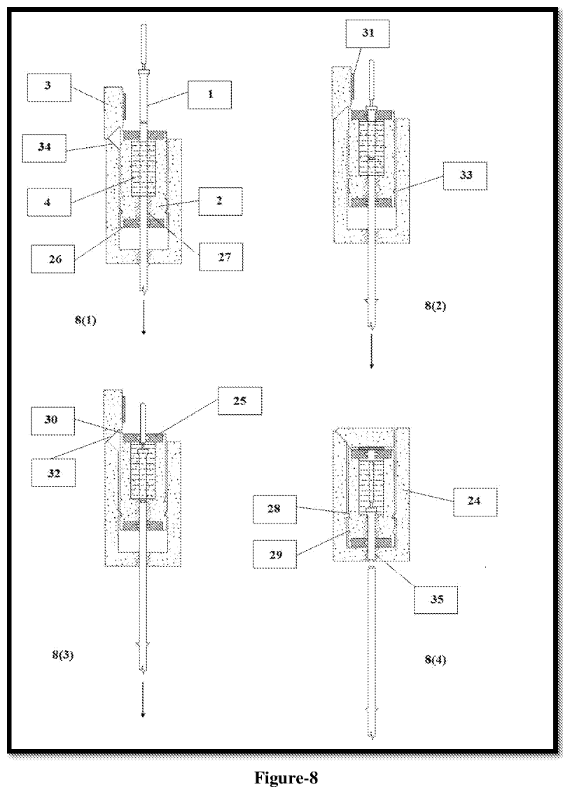

FIG. 8 shows an embodiment where number of parts and assembles are minimized. The embodiment comprises but not limited to a sample container vial [2], an outer triggered housing [24], and a swab [1]. the sample container vial comprises a liquid containing chamber [4], top sealing material [25], bottom sealing material [26], a linear retraction wall [27], top position snap lock [28], and bottom position snap lock [29]. The sample container vial [2] also comprise a sample container unilateral lid [3] which is connected through a lid connecting mechanism [30] including but not limited to snap fit, press fit, leaving hinge, etc. with the sample container vial [2] at the top. The sample container lid have a closing lid sealing material [31] on it for sealing between the lid and the closing lid wall [32]. The swab comprises the same features and component as discussed before. The outer triggered housing comprises a triggered snap lock [33], exit linear retraction wall where the swab can pass through, and a closing guide wall [34].

When the swab is pulled by the user, the swab [1] enters the sample container vial and submerges in the liquid present inside of the sample container liquid chamber, without making any contact with the swab tip as shown in FIG. 8(2) and in FIG. 8(3). Then the detecting and locking slot locks with the sample in the container liquid storage end. At until the point of time the sample container closing lid stays open or stays at vertical position because of the lid locking mechanism and also the triggered snap lock [33] holds the sample container vial [1] by locking with the bottom position snap lock [29]. Because of the excess pulling force, the triggered snap lock [33] loose the holding point of the sample container vial [1] and the sample container vial [1] moves downward along with the swab, the triggered snap lock [33] snaps with or locks with top position snap lock [28]. The top position snap lock [28], bottom position snap lock [29] and the triggered snap lock [33] are designed such that the movement is possible in one way only; also the sample container vial [1] can move only downwards between the distance of top position snap lock [28] and the bottom position snap lock [29] as shown in FIG. 8. The slant section of each position as well as the triggered lock allows to slide after a certain pressure is applied, but the other flat side of each position as well as the triggered lock, are not allowed to move, hence locks it's position.

When the sample container vial travels along with the swab at the distance between the top position snap lock [28] to the bottom position snap lock [29], the closing lid wall [32] comes in contact with the closing guide wall [34]. The lid connecting mechanism allows to rotate the sample container closing lid and allows to seal the container vial from the top. The closing lid and guide wall overlaps such that the opening of the sample container closing lid restrict to open it again. Finally the swab breaks or detaches as shown in FIG. 8(4).

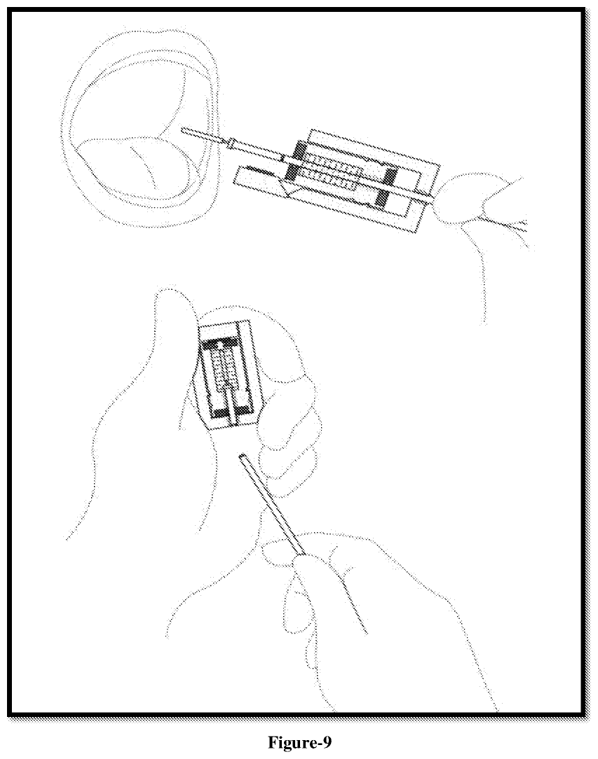

FIG. 9 shows the significant reduction of steps of human interaction compared with conventional method and existing prior art. FIG. 9 is an illustrative view of the steps involved in the operation of the device. The mechanism described herein includes the complete handling of the current invention from the time of sample collection from the body until collected in the container. The mechanism here is also a one-step mechanism which is an embodiment of the current invention.

In FIG. 9 shows the holding position of the device of the present invention, which also is a single unit device for sample collection, extraction and processing. The swab assembled within the container is placed such that swab does not come in contact with other body parts, therefore keeping it contamination free and easy to hold.

In FIG. 9 is the illustrative view of the device after the sample is collected from the desired surface. Herein, after the sample is collected, the swab is pulled down in order to store the sample inside the container. The mechanism here is controlled by a linear one-way retraction movement, allowing the swab to get smoothly pulled inside the container, without coming in contact of any other surface as well as maintain the sealing, closing the sample container lid and breaking the excess swab.

In one embodiment the present invention discloses a method of collection of biological sample in a sample collection vial in any if the proceeding claims, comprising pulling of the swab by an user after collection of sample by the back swab handle [16] using the swab gripper [17]; force opening of the unilateral lid [3] of the sample container vial by the push-base [7]; linear downward movement of the swab with help of linear retraction mechanism [8]; entering of the swab tip in the sample container vial; snap closing of the unilateral lid [3] on reaching of the swab [1] at position where the swab tip [5] is completely submerged within the liquid present inside of the container vial; locking of the triggered snap lock [33] with the top position snap lock [28] to hold the sample container vial [1] by locking at position −2 after the sample is collected by an User. closing of the lid wall [32] with the closing guide wall [34] and sealing of the sample container vial from the top; breaking-off the excessive swab handle automatically the help of the swab breaking mechanism.

Along with sample collection the device is intended for extraction of cells from the swab in an automated fashion as well as integration of passive or active micro fluidic chips in to a predefined section provided for the processing of biological samples. The micro fluidic chip can be integrated within the sample container vial. This micro fluidic chip having a inlet where the extracted cells from the swab can be processed further. The fluid carried along with the extracted cells from the swab can be driven actively or passively inside the micro fluidic chip.

The objective of the sample extraction from the swab is the critical part of the invention. Due to low volume of liquid and high surface area inside of the container vial cause high surface tension. Conventional approaches to extraction of the sample can be challenging.

Conventionally, the extraction of cells from the swab surface is carried out through the use of vortex instrument where by a vibratory mechanical element is used in order to vigorously agitate the vial and its constituents in an attempt to extract cells. Vortex is used in number of defined and commercialized protocols for the collection of genomic DNA from the samples but not limited to Plasmid DNA, but there is a possibility of fragmentation of DNA during vortex step, and results in poor cell/DNA collection. High surface to volume ratio increase difficulties for vortex.

Other common approaches incorporate the use of ultrasonication as a means to agitate the sample liquid and the cells attached onto the surface. Ultrasonication is employed as a rapid cell/DNA extraction tool, but there is a probability of causing the damage to the genomic DNA, but not limited to Plasmid DNA. DNA fragmentation can occur and again, results in poor cell/DNA collection.

These vortex machines and ultrasonication setups are comparatively larger in scale and are difficult to miniaturize without losing their efficiency at extraction of cells from swabs. As well as they are difficult to incorporate in an instrument to allow for an automatic operation. Though these mechanically driven techniques (vortex machines and ultrasonication) are robust for cell lysis, these methods are complex to fabricate and expensive. Therefore, including such systems within a small low cost sample container as the one as is the subject of the invention is extremely difficult if not impossible. Sometimes, mechanical pipetting action is also used to lyse the cells. But it is manually done and adds to the chances of human errors as well as adding a manual step. It can cause the damage to the cell recovered and results in its bad recovery as well as contamination.

Miniaturization of vortex instruments can be carried out through the use of small vibratory actuators which can be built into this container and can be achieved within a low cost constraint. However, small vibratory motors have extremely low output power and are hence significantly less effective at agitating small volumes of liquid in a vial like container. This suggests that if an agitation scheme similar to what is used in a lab environment is to be incorporated within a miniaturized sample container it would pose serious challenges due to the low power output of the small vibratory actuators as well as due to the small volume of liquid that is present in the vial to be agitated. Conventional approaches as mentioned above also are difficult to integrate into cost effective miniaturized sample containers due to the problems stated above.

This is the reason that the use of a pH or heat based approach allows for a simplified mechanical design, miniaturized assembly and a simplified sample extraction system with minimum additional instrumentation required. Improving the geometry of the swab design can also be a key factor to perform the extraction.

Sample extraction mechanism is comprised of thermally actuated or pH dependent reaction allowing for cells to lose their adhesion with the swab because of their thermal or pH responsive behavior in elution buffers. Creation of pH− responsive platform on the swab surface effectively capture/release of cell from the sample. Sample is collected from the buccal (saliva), but not limited to human bloodstains, urine and touched samples with swabs. In doing so cells can be extracted in a simple and efficient manner.

In addition to mechanically driven, but not limited to thermal and chemical (pH based) approaches, other approaches, including the optical, acoustic and electrical are used for the cell lysis. But the optical approach is a cell specific lysis and required a complex instrumentation, which costs more. Acoustic approach have medium range efficiency. Moreover, heat is generated during the cell lysis and it is an expensive technology. Electrical approach is having high efficiency, but required an costly equipment for cell lysis. Therefore, the cell extraction using a thermal or pH dependent approach is a simple way to get the high quality of cell recovery in a cost-effective manner.

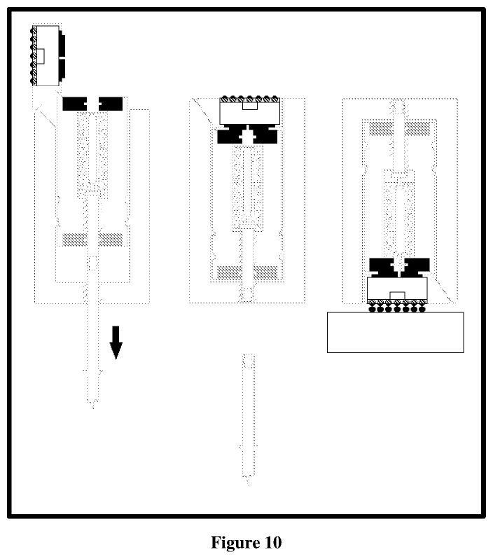

FIG. 10, depicts a micro fluidics that may be placed for processing the sample. In the present embodiment the micro fluidic chip can be placed on the closing lid of the device. The inlet of the micro fluidic device is positioned underneath the lid so that the low amount of fluid along with the extracted sample can flow through actively or passively forces during sample collection.

The micro fluidic device can be placed or fitted with the container closing lid, but not limited to by snap fitted, press fitted, epoxied or glued, over molded, threaded, bolted, welded (liquid welding), etc. As mall liquid flow interface between the micro fluidic chip along with the inlet port through lid sealing material is designed such that the samples along with the fluid can flow smoothly inside of the micro fluidic chip without any obstruction. The sealing is provided by the lid sealing material on both side so that leakages can be completely ruled out.

The micro fluidic chip can be made of but not limited to conventional way like PDMS, Glass, Thermo set or Thermo plastics, Teflon, injection molded, multi stacked additive layer, silicon, etc. This micro fluidic device can be integrated into the sealing cap because the sealing cap may be made of silicon. For detection of the sample as well as for the smart and reliable operation of the micro fluidic chip, not limited to valving of fluid, pumping of fluid, sensing of samples, heating of samples as well as the fluid, etc., of the micro fluidic chip required electronics support. SIP (System in package), WLCSP (Wafer-level chip scale packaging), high density PCB, etc., can be integrated with the micro fluidic chip for fulfill the requirement. The micro fluidic device along with the electronic module can be placed or fitted with the container closing lid, but not limited to by snap fitted, press fitted, epoxied or glued, over molded, threaded, bolted, welded (liquid welding), etc.

In FIG. 10(a) shows the embodiment of the sample collection device with the integrated micro fluidic device along with the electronic module placed in the closing lid of the sample container. In FIG. 10(b) shows after pulling down the swab the mechanism allows to close the container lid, seal the top, extra swab holder breaks apart. In FIG. 10(c) shows how the liquid travels within the micro fluidic device when the device turns around and allows the liquid to came in contact. The small channel present inside of the closing lid sealing allows the liquid enters into the micro fluidic device. The electronics module powered up while in contact with the external signal and power supply module.

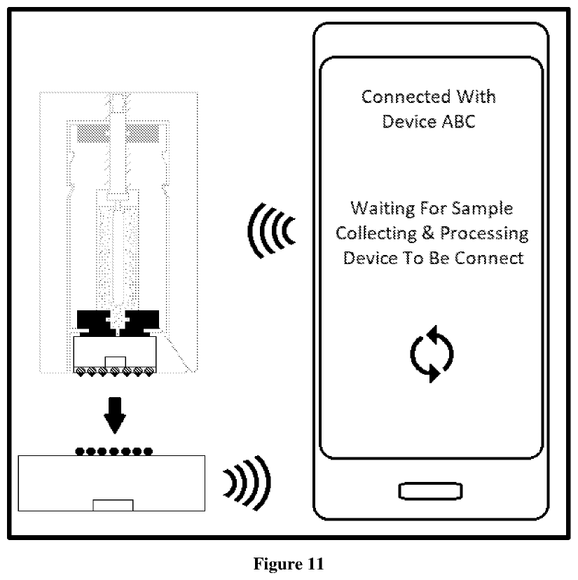

The external signal and power supply module provide power and signal to the electronics module combined with the micro fluidic device, allows to perform the detection and analysis of the task. FIG. 11 show how the external signal and power supply module communicate with another User interface provided electronic module.

The communication between the external signal and power supply module with the User interface provided electronic module can be done but not limited to Bluetooth, BLE (Bluetooth low energy), NFC (Near-field communication), infrared, etc. User can operate the process through the User interface provided electronic module as well as knows the result of the process. The smart device can also provide the status of the process from the beginning till the end.

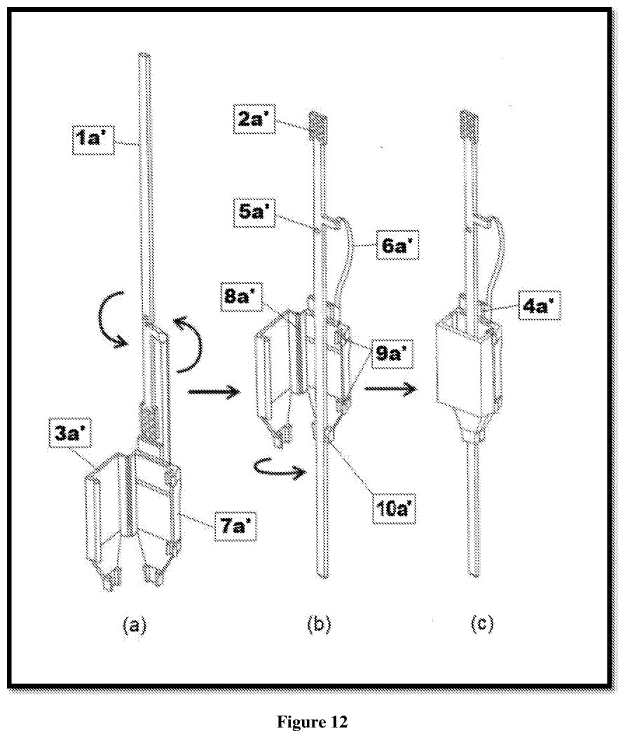

In an additional embodiment of the present invention FIG. 12 shows the steps involved in that current invention which one of these embodiments provides the mechanism of a customized swab [1a′] flipping mechanism. The goal of the invention is to maintain a simple, easy user experience during use of the device as well as be cost effective. The objective of the embodiment as well as the invention is to make the product as a single unit, where the design will be simple and easy to manufacture with all constraints related to the invention.

FIGS. 12(a) to 12(c) is showing the 3 stages of a simple illustrative view of the swabbing mechanism. The single device comprising of customized swab [1a′] designed for collecting samples efficiently compared to others, customized swab tip [2a′], closing lid [4a′], breaking mechanism [5a′], guiding swab-ribbon [6a′], sample collection vial comprises but not limited to front side closing door of the vial [3a′], back side closing door [7a′], snap lock [8a′], living hinge [9a′].

FIG. 12(a) shows the initial state of the device where the product is fabricated as a single part mold. In the next FIG. 12(b) the swab flipped and locked in the swab locking slot [10a′], then locked the closing door of the vial [3a′] to close the vial air tightly. Air will not come across the sample collection vial because of the tight air sealing snap locking. In FIG. 12(c) is an assembled view swab with an air tightly sample collection vial. The one end of the guiding swab-ribbon [6a′] connects with the swab [1a′] and the other end connects with the closing lid [4a′]. The back side closing door [7a′] of the vial connects by living hinges [9a′].

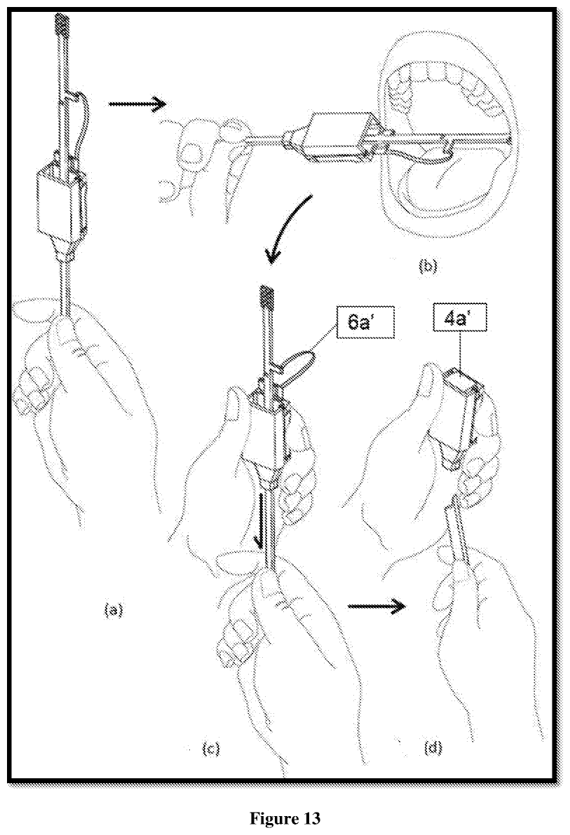

FIG. 13(a) shows correct way to hold the device, FIGS. 13(b) to 13(d) comprises the sample taking process by rubbing the swab tip on the inside wall of the cheek and rubbing it gently, taking out the device, holding the vial with other hand and pulling the swab downwards through one-way linier retraction until the top closing lid [4a′] closes the vial completely. After closing no air can pass the sample collection vial and the vial is totally sealed. The user applies a bit of extra force to break the extra part of the swab from the breaking notch point. During the downward swab pulling the guiding swab-ribbon guides the linear way of the swab also the guiding swab-ribbon [6a′] helps the closing lid to close seal tightly.

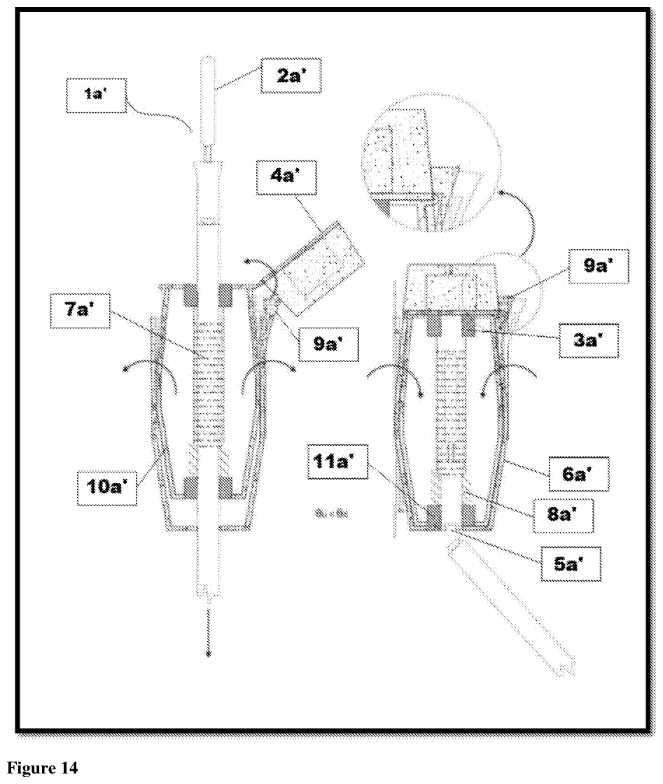

FIG. 14 shows the current invention which one of these embodiments provides the mechanism of a customized swab [1a′] with geometry-based closing lid locking and sealing mechanism described. The goal of the invention also is to maintain a simple, easy user experience during use of the device as well as be cost effective. The objective of the embodiment as well as the invention is to make the product as a single unit, where the design will be simple and easy to manufacture with all constraints related to the invention.

FIGS. 14(a) and 14(b) is showing the 2 stages of a simple illustrative view of the swabbing mechanism and geometry-based closing lid sealing mechanism. The device consists of customized swab [1a′] designed for collecting samples efficiently compared to others, customized swab tip [2a′], closing lid [4a′], breaking mechanism [5a′], geometry based outer enclosure [6a′], sample collection vial [10a′]. Sample collection vial [10a′] comprises but not limited to top sealing material [3a′], bottom sealing material [11a′], liquid storage chamber [7a′], linear retraction wall [8a′]. The geometry based outer enclosure [6a′] comprises but is not limited to lid locking pin [9a′] which locks the closing lid [4a′] after collecting and pulling down the swab into the liquid storage chamber [7a′]. Geometry base outer enclosure [6a′] comprises a lid locking pin [9a′] like a spring and lock mechanism. Initially the lid locking pin [9a′] position opens wide when the sample is collected, after collection of the sample the Sample collection vial [10a′] move in along with the swab and the closing lid comes contact with the lid locking pin [9a′] is closed and sealed at the top of the sample collection vial [10a′], and locked. The lid locking pin [9a′] position closes when the lid is closed. The combination of geometry based outer enclosure [6a′] and the lid locking pin [9a′] the mechanism works together to effect of sealing and closing of sample vial.

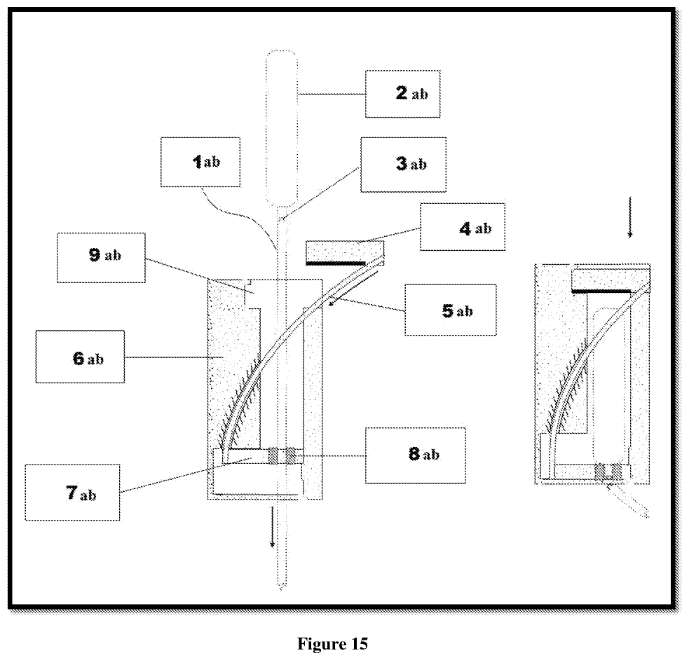

In one embodiment the present invention (FIG. 15) discloses a sample collection device comprising a customised swab [1ab] with sample collection tip [2ab] and breaking mechanism [3ab] which is placed in a liquid contained vial [9ab] so that the swab may linearly retract in downward movement, which further pass through a bottom closing lid [7ab], where the bottom closing lid [7ab] have a bottom closing sealing [8ab]. The bottom closing lid is attached to a flexible ribbon [5ab] which further holds the top closing lid [4ab]. This ribbon passes through the sample collecting and storage vial wall [6ab] and remains outside of sample collecting and storage vial enclosure [9ab] at the initial stage. After completion of the sample collection process, user pulls down the swab [1ab] until it comes in contact with the bottom closing lid [7ab]. The sample collection tip [2ab] pushes the bottom closing lid [7ab] which is assembled with the flexible ribbon [5ab] and top closing lid [4ab], slides downwards and closes the opening of the sample collecting and storage vial enclosure [9ab].The sample collection swab tip [2ab] is pulled completely in until the bottom closing lid [7ab] reaches the bottom surface of the sample collecting and storage vial wall [6ab].Further User applies some force to break the excess part of the swab [1ab] which breaks due to the breaking mechanism [3ab].

While specific language has been used to describe the disclosure, any limitations arising on account of the same are not intended. As would be apparent to a person in the art, various working modifications may be made to the method in order to implement the inventive concept as taught herein. The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

1.-26. (canceled)

27. A sample collection and processing integrate-able device comprising
a. a sample collecting swab [1] comprising a swab tip [5], a swab handle, a swab locking slots [8] and a push-base [7];
b. a sample collection vial [2] comprising liquid for collection of sample [4], a linear guide for swab [9] at the base of the said vial and an unilateral lid [3].

28. The device as claimed in claim 27, wherein the push base [7] of a sample collecting swab [1] enables opening of the unilateral lid [3] only towards and in the sample collection vial [2].

29. The device as claimed in claim 27, wherein the unilateral lid [3] which is connected with the sample container vial [2] at the top through a lid connecting mechanism [30] selected from a group comprising snap fit, press fit, leaving hinge.

30. The device as claimed in claim 27, wherein the sample collecting swab [1] pierces and extends through the base of the sample collection vial [2], and
the sample collecting swab [1] movement in reverse or backward direction or any leakage of the liquid in the vial is prevented by one way linear retraction mechanism, wherein the one way linear retraction mechanism comprises a locking key bar [10] connected to a locking spring [11], and sealing material [12].

31. The device as claimed in claim 27, wherein a micro fluidic chip [35] is positioned underneath the lid along with the inlet port such that the samples along with the fluid can flow smoothly inside of the micro fluidic chip [35] without any obstruction, and wherein
the microfluidic chip [35] is connected to an external electronic device such as a mobile phone, laptop, and can store data in a server.

32. The device as claimed in claim 27, wherein the sample collection vial [2] further comprises an outer triggered housing [24], top sealing material [25], bottom sealing material [26], top position snap lock [28], and bottom position snap lock [29], closing lid sealing material [31].

33. The device as claimed in claim 32, wherein the outer triggered housing comprises a triggered snap lock [33], exit linear retraction wall, and a closing guide wall [34].

34. The device as claimed in claim 27, wherein the triggered snap lock [33] holds the sample container vial [1] by locking with the bottom position snap lock [29] at position −1 before the sample is collected by an User, and
the triggered snap lock [33] loose the holding point with bottom position snap lock [29] on being pulled by the user, the sample container vial [1] moves downward along with the swab and the triggered snap lock [33] snaps with or locks with top position snap lock [28] at position −2 after sample collection by an User.

35. The device as claimed in claim 27, wherein the closing lid wall [32] comes in contact with the closing guide wall [34] and seals the sample container vial from the top.

36. The device as claimed in claim 27, wherein the closing lid and guide wall overlaps such that the opening of the sample container closing lid restrict to open it again.

37. The device as claimed in claim 30, wherein the locking spring [11] allows the locking key bar [10] locks the sample collection swab [1] when in contact with swab locking slots [8].

38. The device as claimed in claim 27, wherein the sample collecting swab comprises of a swab tip sub assembly having a swab tip, a swab collector flexible linkage [13], a front swab handle [14], and a swab handle sub assembly having back swab handle [16], swab gripper [17], retraction notch [18], and back swab breaking mechanism [19].

39. The device as claimed in claim 38, wherein the front swab handle [14] further comprises front swab detecting and locking slot [15], and front swab breaking mechanism [16].

40. The device as claimed in claim 38, wherein the swab tip sub assembly and the swab handle sub assembly connect together with the help of the swab breaking mechanism.

41. The device as claimed in claim 38, wherein the swab detecting and locking slot [15] locks at the bottom surface of the sample container liquid storage chamber by the retraction notch [18].

42. The device as claimed in claim 38, wherein the swab handle breaks at front swab breaking mechanism [16].

43. The device as claimed in claim 31, wherein the micro fluidic chip [35] is made from material selected from a group comprising PDMS, Glass, thermoset or thermo plastics, Teflon, injection molded, multi stacked additive layer, silicon, and
the micro fluidic chip [35] senses the biological sample by a methods selected from a group comprising valving of fluid, pumping of fluid, sensing of samples, heating of samples and is equipped with. SIP (System in package), WLCSP (Wafer-level chip scale packaging), high density PCB.

44. A method of collection of biological sample in a sample collection vial by the sample collection and processing integrate-able device as claimed in claim 38, comprising;
a. pulling of the swab by an user after collection of sample by the back swab handle [16] using the swab gripper [17];
b. force opening of the unilateral lid [3] of the sample container vial by the push-base [7];
c. linear downward movement of the swab with help of a linear retraction mechanism [8];
d. entering of the swab tip in the sample container vial;
e. snap closing of the unilateral lid [3] on reaching of the swab [1] at position where the swab tip [5] is completely submerged within the liquid present inside of the container vial;
f. locking of the triggered snap lock [33] with the top position snap lock [28] to hold the sample container vial [1] by locking at position −2 after the sample is collected by an User;
g. closing of the lid wall [32] with the closing guide wall [34] and sealing of the sample container vial from the top;
h. breaking-off the excessive swab handle automatically the help of the swab breaking mechanism.

45. A sample collection single-mold device comprising;
a. swab [1a′] designed for collecting samples comprising swab tip [2a′];
b. closing lid [4a′];
c. breaking mechanism [5a′];
d. guiding swab-ribbon [6a′];
e. sample collection vial comprising front closing door of the vial [3a′], back closing door [7a′], snap lock [8a′], living hinge [9a′].

46. A sample collection device comprising;
a. customised swab [1ab] comprising sample collection tip [2ab];
b. breaking mechanism [3ab] enabled by linear retraction in the downward movement of the swab;
c. a vial containing the liquid [9ab];
d. a bottom closing lid [7ab], comprising bottom closing sealing [8ab]; top closing lid [4ab];
e. a flexible ribbon [5ab] annexed to bottom closing lid [7ab] and the top closing lid [4ab],
wherein the flexible ribbon [5ab] passes through the sample collecting and storage vial wall [6ab] and remains outside the sample collecting and storage vial enclosure [9ab] before the collection of sample and is sealed within the sample collecting and storage vial wall [6ab] after collection of sample.
